(19) United States
(12) Patent Application Publication
(10) Pub. No.: US 2008/0003134 A1
(43) Pub. Date: Jan. 3, 2008

(54) METHODS OF INHIBITING THE DISTORTIONS THAT OCCUR DURING THE PRODUCTION OF SILICONE HYDROGEL OPHTHALMIC LENSES

(76) Inventors: James D. Ford, Orange Park, FL (US); Azaam Alli, Jacksonville, FL (US); Dharmesh Dubey, Jacksonville, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/764,846

(22) Filed: Jun. 19, 2007

Related U.S. Application Data
(60) Provisional application No. 60/817,728, filed on Jun. 30, 2006.

(51) Int. Cl. A61L 2/02

(52) U.S. Cl. 422/28

(57) ABSTRACT

Methods of reducing the distortion of silicone hydrogel lenses during sterilization are disclosed.

### RELATED APPLICATIONS

This application is a non-provisional filing of U.S. Patent Application No. 60/817,728, filed on Jun. 30, 2006, a provisional patent application.

This invention related to methods of reducing distortions that occur during the production of silicone hydrogel contact lenses.

### BACKGROUND

Contact lenses have been used commercially to improve vision since the 1950s. The first contact lenses were made of hard materials. Although these lenses are currently used, they are not suitable for all patients due to their poor initial comfort. Later developments in the field gave rise to soft contact lenses, based upon hydrogels, which are extremely popular today. Further developments have lead to the development of silicone hydrogel lenses. Such silicone hydrogel lenses have are known higher oxygen permeabilities and such are often more comfortable to wear than contact lenses made of hard materials. However, silicone hydrogel lenses are not without problems.

Silicone hydrogel lenses may be creased or otherwise physically distorted during their production. This problem can be seen when the lenses are sterilized. Typically, silicone hydrogel lenses are packaged in individual container containing ophthalmic packaging solutions and heated to temperatures of about 100° C. or greater. During this process silicone hydrogels are know to adhere to their packaging materials or be otherwise distorted. One approach to this issue is to add certain surfactants to the ophthalmic packaging solutions another approach is to modify the inner surface of the package. Even though these approaches exist, it is preferable to expand the range of solutions for this problem. This invention offers an alternative solution to the problem of distortion of silicone hydrogels during their production.

### BRIEF DESCRIPTION OF THE DRAWING

FIG. 1 An ophthalmic lens package in a horizontal orientation.

### DETAILED DESCRIPTION OF THE INVENTION

This invention includes a method of inhibiting the distortion of silicone hydrogel ophthalmic lenses during sterilization comprising, consisting essentially of, or consisting of sterilizing said silicone hydrogel ophthalmic lenses in a package, wherein said package is oriented in a substantially horizontal position.

As used herein “silicone hydrogel ophthalmic lenses” refers to ophthalmic devices that resides in or on the eye. These devices can provide optical correction or may be cosmetic. The term lens includes but is not limited to soft contact lenses, intraocular lenses, overlay lenses, ocular inserts, and optical inserts. The preferred lenses of the invention are silicone hydrogel lenses described in U.S. Pat. No. 5,710,302, WO 9421698, EP 406161, JP 2000016905, U.S. Pat. No. 5,998,498, U.S. patent application Ser. No. 09/532,943, U.S. Pat. No. 6,087,415, U.S. Pat. No. 5,760,100, U.S. Pat. No. 5,776,999, U.S. Pat. No. 5,789,461, U.S. Pat. No. 5,849,811, and U.S. Pat. No. 5,965,631. The foregoing references are hereby incorporated by reference in their entirety. The preferred lenses include but are not limited to silicone hydrogels such as acquafilcon A, balafilcon A, lotrafilcon A, and silicone hydrogels as prepared in U.S. Pat. No. 5,998,498, U.S. patent application Ser. No. 09/532,943, a continuation-in-part of U.S. patent application Ser. No. 09/532,943, filed on Aug. 30, 2000, U.S. Pat. No. 6,087,415, U.S. Pat. No. 5,760,100, U.S. Pat. No. 5,776,999, U.S. Pat. No. 5,789,461, U.S. Pat. No. 5,849,811, and U.S. Pat. No. 5,965,631. The most preferred silicone hydrogel ophthalmic lenses are acquafilcon A. These patents as well as all other patents disclosed in this application are hereby incorporated by reference in their entirety.

As used herein sterilizing refers to heating silicone hydrogel ophthalmic lenses at temperatures and times required to kill microorganisms. Preferably temperatures are greater than about 100° C. and times are greater than about 15 minutes. As discussed previously, silicone hydrogel ophthalmic lenses are sterilizing in ophthalmic packaging solutions. The ophthalmic packing solutions of the invention may be any water-based solution that is used for the storage of contact lenses. Typical solutions include, without limitation, saline solutions, other buffered solutions, and deionized water. The preferred aqueous solution is saline solution containing salts including, without limitation, sodium chloride, sodium borate, sodium phosphate, sodium hydrogenphosphate, sodium dihydrogenphosphate, or the corresponding potassium salts of the same. These ingredients are generally combined to form buffered solutions that include an acid and its conjugate base, so that addition of acids and bases cause only a relatively small change in pH. The buffered solutions may additionally include 2-(N-morpholino)ethanesulfonic acid (MES), sodium hydroxide, 2,2-bis(hydroxymethyl)-2,2′,2″-nitrilotriethanol, n-tris(hydroxymethyl)methyl-2-aminoethanesulfonic acid, citric acid, sodium citrate, sodium carbonate, sodium bicarbonate, acetic acid, sodium acetate, ethylenediamine tetraacetic acid and the like and combinations thereof. Preferably, the solution is a borate buffered or phosphate buffered saline solution.

“Package” refers to any container that may be used to sterilize silicone hydrogel ophthalmic lens. Examples of such containers are disclosed in the following publications, U.S. Pat. Nos. D435,966 S; 4,691,820; 5,467,868; 5,704,468; 5,823,327; 6,050,398, which are hereby incorporated by reference in their entirety.

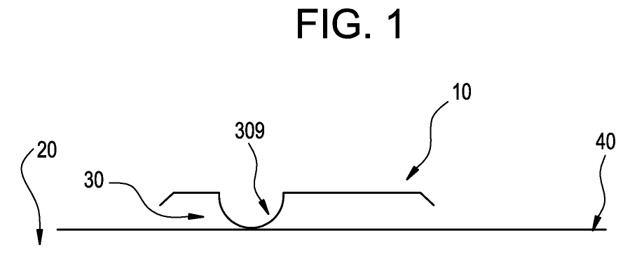

“Horizontal” refers to the position in which the package sits during sterilization. Referring to FIG. 1, the orientation of the flange 10 of the package 20 is horizontal with respect to the level surface of table top 40. During sterilization the silicone hydrogel ophthalmic lens (not shown) rests in bowl 30 where the convex surface of the silicone hydrogel ophthalmic lens is adjacent to the concave surface of bowl 30 (not shown).

### EXAMPLE 1

Solution A with a Variety of Surfactants

Etafilcon A hydrogel and acquafilcon A contact lenses were placed in individual polypropylene contact lens packages. Saline packaging solution was added to each package to cover each lens and the package was sealed. Half of the packages were placed in a holder in the vertical position and the other half were placed in a holder in the horizontal position. The packages were heated to 121° C. for about 20 minutes. The packages were cooled and opened and the lenses were visually examined. The etafilcon A lenses sterilized in both the horizontal and vertical positions maintained their shape without folds creases or other deformities. Acquafilcon A lenses that sterilized in a vertical orientation contained creases and other deformities. Acquafilcon A lenses that were sterilized in a horizontal position did not contain such deformities.

What is claimed is;

1. A method of inhibiting the distortion of silicone hydrogel ophthalmic lenses during sterilization comprising sterilizing said silicone hydrogel ophthalmic lenses in a package, wherein said package is oriented in a substantially horizontal position.

2. The method of claim 1 wherein the silicone hydrogel ophthalmic lens is selected from the group consisting of silicone hydrogels as prepared in U.S. Pat. No. 5,998,498, U.S. patent application Ser. No. 09/532,943, a continuation-in-part of U.S. patent application Ser. No. 09/532,943, filed on Aug. 30, 2000, U.S. Pat. No. 6,087,415, U.S. Pat. No. 5,760,100, U.S. Pat. No. 5,776,999, U.S. Pat. No. 5,789,461, U.S. Pat. No. 5,849,811, and U.S. Pat. No. 5,965,631.

3. The method of claim 1 wherein the silicone hydrogel ophthalmic lens is selected from the group consisting of acquafilcon A, balafilcon A, and lotrafilcon A.

4. The method of claim 1 wherein the silicone hydrogel ophthalmic lens is acquafilcon A.

5. The method of claim 1 wherein the sterilization occurs at a temperature of greater than about 100° C.

6. The method of claim 1 wherein the sterilization occurs at a temperature of greater than about 120° C.

7. The method of claim 1 wherein sterilization occurs in about 10 minutes.

8. The method of claim 1 wherein sterilization occurs in about 20 minutes.

9. The method of claim 1 wherein sterilization occurs in about 18 to about 20 minutes at a temperature of about 118° C. to about 120° C.

10. The method of claim 9 wherein the silicone hydrogel ophthalmic lens is acquafilcon A.
